(19) United States
(12) Patent Application Publication
(10) Pub. No.: US 2018/0008161 A1
(43) Pub. Date: Jan. 11, 2018

(54) METHOD FOR RECOGNIZING IRIS BASED ON USER INTENTION AND ELECTRONIC DEVICE FOR THE SAME

(71) Applicant: Samsung Electronics Co., Ltd., Gyeonggi-do (KR)

(72) Inventors: Hyung-Woo SHIN, Seoul (KR); Hyemi LEE, Seoul (KR); Hyung Min LEE, Seoul (KR)

(21) Appl. No.: 15/638,578

(22) Filed: Jun. 30, 2017

(30) Foreign Application Priority Data
Jul. 8, 2016 (KR) 10-2016-0086746

(51) Int. Cl. A61B 5/0476; G02B 27/01; A61B 5/00; G06K 9/00; G02B 27/00

(52) U.S. Cl. CPC A61B 5/0476; G02B 27/0172; A61B 5/6887; G02B 27/00; G06K 9/00

(57) ABSTRACT

A method and an apparatus include a display, an iris scanning sensor, and a processor functionally coupled with the display, and the iris scanning sensor, wherein the processor activates the iris scanning sensor when receiving a display-on event in a display-off state that is an intended user input.

### CLAIM OF PRIORITY

This application claims priority under 35 U.S.C. §119(a) to Korean Patent Application Serial No. 10-2016-0086746, which was filed in the Korean Intellectual Property Office on Jul. 8, 2016, the content of which is incorporated herein by reference.

### BACKGROUND

### 1. Field of the Disclosure

The present disclosure relates generally to a method and an electronic device for recognizing an iris based on a user intention.

### 2. Description of the Related Art

With recent advances in digital technology, various electronic devices such as mobile communication terminals, Personal Digital Assistants (PDAs), digital organizers, smart phones, Tablet Personal Computers (PCs), and wearable devices are widely used. To support and enhance functionality, hardware and/or software of the electronic devices are improved over time. However, with the recent advances in digital technology, it is important to secure electronic devices to protect the user's privacy and data.

### SUMMARY

According to various embodiments, an electronic device may include a display, an iris scanning sensor, and a processor functionally coupled with the display and the iris scanning sensor, wherein the processor activates the iris scanning sensor when receiving a display-on event in a display-off state that is an intended user input.

According to various embodiments, a method for operating an electronic device which includes an iris scanning sensor, may include detecting a display-on event in a display-off state, and when the display-on event is a user intended input, activating the iris scanning sensor.

An electronic device comprising a touchscreen; an infrared camera; a processor operably coupled to the infrared camera and the touchscreen, the processor configured to: when the touchscreen is de-illuminated, detect one of a button selection, a touch on the touchscreen, and receipt of a push notification; causing the touchscreen to illuminate responsive to detecting one of a button selection, a touch on the touchscreen, and receipt of the push notification and display a locked screen; when detecting a button selection or a touch on the touchscreen, automatically activating the infrared camera; when detecting a push notification, displaying a prompt on the touchscreen on the touchscreen for a user input requesting iris detection.

According to various embodiments, by recognizing an iris based on a user intention, unnecessary light source module (e.g., LED) flickering or unintentional electronic device unlocking can be prevented.

According to various embodiments, by recognizing an iris based on a user intention, user inconvenience caused by unnecessary light source module (e.g., LED) flickering can be avoided.

According to various embodiments, security of the electronic device can be enhanced by blocking IR camera activation from unlocking the electronic device regardless of a user's intention.

According to various embodiments, unnecessary power consumption can be reduced by preventing the light source module and the IR camera from activating for the iris recognition regardless of a user's intention.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments of the present disclosure will be more apparent from the following description taken in conjunction with the accompanying drawings, in which:

FIG. 1 is a block diagram of an electronic device according to various embodiments;

FIG. 2 is a flowchart of an operating method of an electronic device according to various embodiments;

FIG. 3A and FIG. 3B are diagrams of a user interface for iris recognition according to various embodiments;

FIG. 4 is a flowchart of an iris recognition method of an electronic device according to various embodiments;

FIG. 5 is a diagram of a user interface for user authentication according to various embodiments;

FIG. 6 and FIG. 7 are diagrams of a user interface based on a user input according to various embodiments;

FIG. 8 is a flowchart of an iris recognition method of an electronic device according to various embodiments; and

FIG. 9 is a diagram of another user interface based on a user intention according to various embodiments.

Throughout the drawings, like reference numerals will be understood to refer to like parts, components and structures.

### DETAILED DESCRIPTION

Hereinafter, various embodiments of the present disclosure are described with reference to the accompanying drawings. However, it should be understood that there is no intent to limit the present disclosure to the particular forms disclosed herein; rather, the present disclosure should be construed to cover various modifications, equivalents, and/or alternatives of the embodiments of the present disclosure. In the description below of the accompanying drawings, similar reference numerals can be used to designate similar elements. The various embodiments included in this disclosure are presented for the explanation and the understanding of the technical contents, not to limit the scope of the invention. Accordingly, the scope of the present disclosure should be construed to include all the changes or other various embodiments based on technical ideas of the present disclosure.

An electronic device according to an embodiment of the present disclosure can include any device using one or more of various processors such as an Application Processor (AP), a Communication Processor (CP), a Graphics Processing Unit (GPU), and a Central Processing Unit (CPU), such as any information communication device, multimedia device, wearable device, and their application devices, supporting a function (e.g., a communication function, a displaying function) according to various embodiments of the present disclosure.

An electronic device according to an embodiment of the present disclosure can include at least one of, for example, a smartphone, a tablet Personal Computer (PC), a mobile phone, a video phone, an e-book reader, a desktop PC, a laptop PC, a netbook computer, a workstation, a server, a Personal Digital Assistant (PDA), a Portable Multimedia Player (PMP), a Moving Picture Experts Group Audio Layer 3 (MP3) player, a mobile medical appliance, a camera, and a wearable device (e.g., smart glasses, a Head-Mounted-Device (HMD), or a smart watch).

An electronic device according to an embodiment of the present disclosure can be a smart home appliance. The smart home appliance can include at least one of, for example, a television, a Digital Video Disk (DVD) player, a refrigerator, an air conditioner, a vacuum cleaner, a washing machine, a set-top box, a home automation control panel, a Television (TV) box (e.g., Samsung HomeSync™, Apple TV™, or Google TV™), a game console (e.g., Xbox™, PlayStation™), and an electronic frame. Also, the electronic device can include at least one of a navigation device and an Internet of Things (IoT) device.

In various embodiments, an electronic device can combine one or more of those various devices. The electronic device can be a flexible device. The electronic device is not limited to the foregoing devices and can include a newly developed electronic device.

The term “user” can refer to a person using an electronic device or a device using an electronic device (e.g., an artificial intelligence electronic device). In an embodiment of the present disclosure, a module or a program module can further include at least one or more of the aforementioned components, or omit some of them, or further include additional other components. Operations performed by modules, program modules, or other components according to various embodiments of the present disclosure can be executed in a sequential, parallel, repetitive, or heuristic manner. In addition, some of the operations can be executed in a different order or be omitted, or other operations can be added.

In certain electronic devices, a user can take a picture using a camera embedded in the electronic device without having to use a separate camera, find directions from their location to a destination using a Global Positioning System (GPS) module of the electronic device without having to use a separate navigation system, and make a payment using the electronic device without cash or a credit card. The electronic device enhances user convenience, however, it is desirable to protect the user's private information because various personal information (e.g., names, phone numbers, addresses, photos, contacts, etc.) that are stored in the electronic device. To reinforce the security, the electronic device can protect the user's private information using an iris recognition function.

When the electronic device is configured to unlock using the iris recognition and its display is turned on, the electronic device automatically enables an Infrared (IR) camera for the iris recognition. For example, when certain events occur, such as receiving push message, or connecting a charger, etc.), the electronic device can automatically and inadvertently turn on the display . Turning on the display can flicker a Light Emitting Diode (LED) for the iris recognition or unlock by recognizing an iris using the IR camera. That is, LED flickering due to inadvertently turning on the display can cause discomfort to the user. Also, unlocking the electronic device unintentionally can compromise the security of the electronic device. Further, the electronic device can consume unnecessary power to operate the LED and the IR camera.

Now, a method and an apparatus for recognizing an iris based on a user intention are explained according to an embodiment of the present disclosure. However, various embodiments of the present disclosure are not restricted by or limited to contents which will be described below and therefore, and it should be noted that they may be applied to various embodiments based on the embodiments which will be described below. In embodiments of the present disclosure described below, a hardware approach will be described as an example. However, since the embodiments of the present disclosure include a technology using both hardware and software, the present disclosure does not exclude a software-based approach.

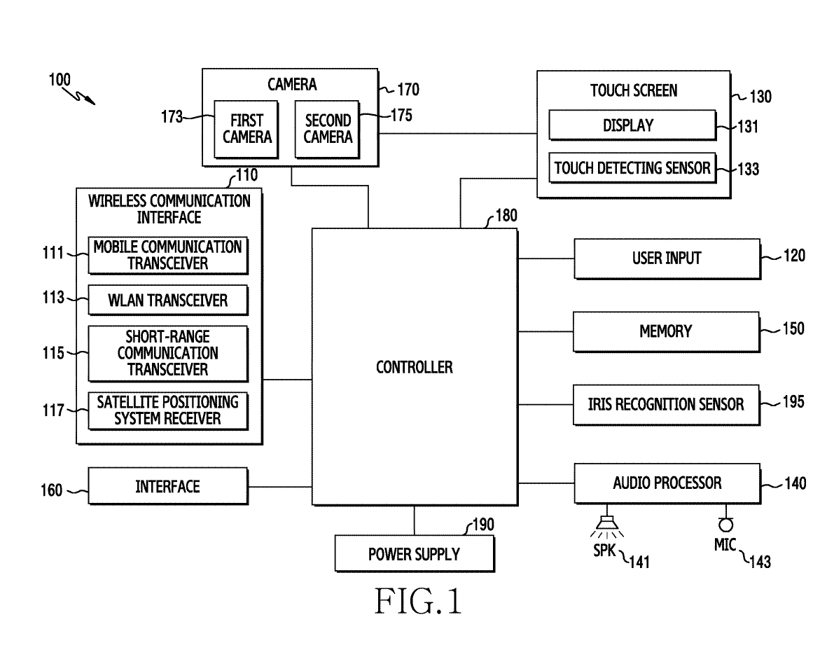

FIG. 1 is a block diagram of an electronic device according to various embodiments.

Referring to FIG. 1, an electronic device 100 according to various embodiments of the present disclosure can include a wireless Communication Interface 110, a user input 120, a touch screen 130, an audio processor 140, a memory 150, an interface 160, a camera 170, a controller 180, a power supply 190, and an IRIS recognition sensor 195. The electronic device 100 can include more components (e.g., a biometric recognition module (e.g., a fingerprint recognition module), an illuminance sensor, a front camera, etc.) or less components than those in FIG. 1. For example, the electronic device 100 may not include, according to its type, some components such as wireless communication interface 110. The components of the electronic device 100 can be mounted inside or outside a housing (or a main body) of the electronic device 100.

According to various embodiments of the present disclosure, the display 131 can display (output) various information processed in the electronic device 100. For example, the display 131 can display a first user interface and a second user interface for the iris recognition, a user interface for user authentication, a user interface or a Graphical User Interface (GUI) based on a user input.

The wireless communication interface 110 can include one or more modules enabling wireless communication between the electronic device 100 and another external electronic device. The wireless communication interface 110 can include a module (e.g., a short-range communication module, a long-range communication module) for communicating with the external electronic device in vicinity. For example, the wireless communication interface 110 can include a mobile communication transceiver 111, a Wireless Local Area Network (WLAN) transceiver 113, a short-range communication transceiver 115, and a satellite positioning system receiver 117.

The mobile communication transceiver 111 can send and receive radio signals to and from at least one of a base station, the external electronic device, and various servers (e.g., an integration server, a provider server, a content server, an Internet server, or a cloud server) over a mobile communication network. The radio signals can include a voice signal, a data signal, or various control signals. The mobile communication transceiver 111 can send various data required for operations of the electronic device 100 to an external device (e.g., a server or another electronic device) in response to a user request. In various embodiments, the mobile communication module 111 can send and receive radio signals based on various communication schemes. For example, the communication schemes can include, but not limited to, Long Term Evolution (LTE), LTE Advanced (LTE-A), Global System for Mobile communication (GSM), Enhanced Data GSM Environment (EDGE), Code Division Multiple Access (CDMA), Wideband CDMA (WCDMA), Universal Mobile Telecommunications System (UMTS), and Orthogonal Frequency Division Multiple access (OFDMA).

The WLAN transceiver 113 can indicate a transceiver for establishing wireless Internet access and a WLAN link with the other external electronic device. The WLAN transceiver 113 can be embedded in or mounted outside the electronic device 100. The wireless Internet technique can employ Wireless Fidelity (WiFi), Wireless broadband (Wibro), World interoperability for Microwave Access (WiMax), High Speed Downlink Packet Access (HSDPA), or millimeter Wave (mmWave). In association with the other external electronic device connected with the electronic device 100 over a network (e.g., wireless Internet network), the WLAN transceiver 113 can send or receive various data of the electronic device 100 to or from the outside (e.g., the external electronic device or the server). The WLAN transceiver 113 can keep turning on, or be turned on according to setting of the electronic device 100 or a user input.

The short-range communication transceiver 115 can indicate a transceiver for conducting short-range communication. The short-range communication can employ Bluetooth, Bluetooth Low Energy (BLE), Radio Frequency Identification (RFID), Infrared Data Association (IrDA), Ultra Wideband (UWB), Zigbee, or Near Field Communication (NFC). In association with the other external electronic device (e.g., an external sound device) connected with the electronic device 100 over a network (e.g., a short-range communication network), the short-range communication transceiver 115 can send or receive various data of the electronic device 100 to or from the outside. The short-range communication transceiver 115 can keep turning on, or be turned on according to the setting of the electronic device 100 or a user input.

The satellite positioning system receiver 117 is a transceiver for acquiring a location of the electronic device 100. For example, the satellite positioning system receiver 117 can include a receiver for receiving GPS signals. The satellite positioning system receiver 117 can measure the location of the electronic device 100 using triangulation. For example, the satellite positioning system receiver 117 can calculate distance information and time information from three or more base stations, apply the triangulation to the calculated information, and thus calculate current three-dimensional location information based on latitude, longitude, and altitude. Alternatively, the satellite positioning system receiver 117 can calculate the location information by continuously receiving location information of the electronic device 100 from four or more satellites in real time. The location information of the electronic device 100 can be acquired in various manners.

The user input 120 can generate input data for controlling the electronic device 100 in response to a user input. The user input 120 can include at least one input means for detecting user's various inputs. For example, the user input 120 can include a key pad, a dome switch, a physical button, a (resistive/capacitive) touch pad, a joystick, and a sensor. Part of the user input 120 can be implemented as a button outside the electronic device 100, and part or whole of the user input 120 may be implemented as a touch panel. The user input 120 can receive a user input for initiating an operation of the electronic device 100, and generate an input signal according to the user input according to various embodiments of the present disclosure.

The touch screen 130 indicates an input/output device for executing an input function and a displaying function at the same time, and can include a display 131 and a touch sensor 133. The touch screen 130 can provide an input/output interface between the electronic device 100 and the user, forward a user's touch input to the electronic device 100, and serve an intermediary role for showing an output from the electronic device 100 to the user. The touch screen 130 can display a visual output to the user. The visual output can include text, graphics, video, and their combination. The touch screen 130 can display various screens according to the operations of the electronic device 100 through the display 131. As displaying a particular screen on the display 131, the touch screen 130 can detect an event (e.g., a touch event, a proximity event, a hovering event, an air gesture event) based on at least one of touch, hovering, and air gesture from the user through the touch sensor 133, and send an input signal of the event to the controller 180.

The display 131 can support a screen display in a landscape mode, a screen display in a portrait mode, or a screen display according to transition between the landscape mode and the portrait mode, based on a rotation direction (or an orientation) of the electronic device 100. The display 131 can employ various displays. The display 131 can employ a flexible display. For example, the display 131 can include a bent display which can be bent or rolled without damages by use of a thin and flexible substrate like paper.

The bent display can be coupled to a housing (e.g., a main body) and maintain its bent shape. The electronic device 100 may be realized using a display device which can be freely bent and unrolled like a flexible display as well as the bended display. The display 131 can exhibit foldable and unfoldable flexibility by substituting a glass substrate covering a liquid crystal with a plastic film in a Liquid Crystal Display (LCD), a Light Emitting Diode (LED) display, an Organic LED (OLED) display, an Active Matrix OLED (AMOLED) display, or an electronic paper. The display 131 can be extended and coupled to at least one side (e.g., at least one of a left side, a right side, an upper side, and a lower side) of the electronic device 100.

The touch sensor 133 can be disposed in the display 131, and detect a user input for contacting or approaching a surface of the touch screen 130. The touch sensor 133 can receive the user input for initiating the operation to use the electronic device 100 and issue an input signal according to the user input. The user input includes a touch event or a proximity event input based on at least one of single-touch, multi-touch, hovering, and air gesture input. For example, the user input can be input using tap, drag, sweep, swipe, flick, drag and drop, or a drawing gesture (e.g., writing).

The audio processor 140 can send to a speaker (SPK) 141 an audio signal input from the controller 180, and forward an audio signal such as a voice input from a microphone (MIC) 143 to the controller 180. The audio processor 140 can convert and output voice/sound data into an audible sound through the speaker 141 under control of the controller 180, and convert an audio signal such as a voice received from the microphone 143 into a digital signal to forward the digital signal to the controller 180. The audio processor 140 can output an audio signal responding to the user input according to audio processing information (e.g., an effect sound, a music file, etc.) inserted into data.

The speaker 141 can output audio data received from the wireless communication interface 110 or stored in the storage unit 150. The speaker 141 may output sound signals relating to various operations (functions) in the electronic device 100. Although not depicted, the speaker 141 can include an attachable and detachable earphone, headphone, or headset, connected to the electronic device 100 through an external port.

The microphone 143 can receive and process an external sound signal into electric voice data. Various noise reduction algorithms can be applied to the microphone 143 in order to eliminate noises generated in the received external sound signal. The microphone 143 can receive an audio stream such as a voice command (e.g., a voice command for initiating a music application). The microphone 143 can include an internal microphone built in the electronic device 100 and an external microphone connected to the electronic device 100.

The memory 150 can store one or more programs executed by the controller 180, and may temporarily store input/output data. The input/output data can include, for example, video, image, photo, and audio files. The memory 150 can store the obtained data obtained, store the data obtained in real time in a temporary storage device, and store data to store in a storage device allowing for long-term storage.

The memory 150 can store instructions for detecting a display-on event in a display-off state, determining whether the display-on event is intended by the user, and activating an iris scanning sensor when the display-on event is intended by the user. In various embodiments, the memory 150 can store instructions for, when executed, causing the controller 180 (e.g., one or more processors) to detect a display-on event in a display-off state, to determine whether the display-on event is intended by the user, and to activate the iris scanning sensor when the display-on event is intended by the user. The memory 150 can permanently or temporarily store an Operating System (OS) of the electronic device 100, a program relating to the input and the display control using the touch screen 130, a program for controlling various operations (functions) of the electronic device 100, and various data generated by the program operations.

The memory 150 can include an extended memory (e.g., an external memory) or an internal memory. The memory 150 can include at least one storage medium of a flash memory type, a hard disk type, a micro type, a card type memory (e.g., a Secure Digital (SD) card or an eXtreme Digital (XD) card), a Dynamic Random Access Memory (DRAM), a Static Random Access Memory (SRAM), a Read-Only Memory (ROM), a Programmable ROM (PROM), an Electrically Erasable Programmable ROM (EEPROM), a Magnetic RAM (MRAM), a magnetic disc, and an optical disc type memory. The electronic device 100 may operate in association with a web storage which serves as a storage of the memory 150 on the Internet.

The memory 150 can store various software programs. For example, software components can include an OS software module, a communication software module, a graphic software module, a user interface software module, a MPEG module, a camera software module, and one or more application software modules. The module which is the software component can be represented as a set of instructions and accordingly can be referred to as an instruction set. The module may be referred to as a program.

The OS software module can include various software components for controlling general system operations. Such general system operation control can include, for example, memory management and control, and power control and management. The OS software module can also process normal communication between various hardware (devices) and software components (modules).

The communication software module can enable communication with another electronic device, such as a computer, a server, or a portable terminal, through the wireless communication interface 110. Also the communication software module can be configured in a protocol structure corresponding to its communication method. The graphic software module can include various software components for providing and displaying graphics on the touch screen 130. The term ‘graphics’ can encompass a text, a webpage, an icon, a digital image, a video, and an animation.

The user interface software module can include various software components relating to the user interface. For example, the user interface software module is involved in a status change of the user interface and a condition for the user interface status change. The MPEG module can include a software component enabling digital content (e.g., video, audio), processes and functions (e.g., content creation, reproduction, distribution, and transmission). The camera software module can include camera related software components allowing camera related processes and functions.

The application module can include a web browser including a rendering engine, e-mail, instant message, word processing, keyboard emulation, address book, widget, Digital Right Management (DRM), iris scan, context cognition, voice recognition, and a location based service.

The interface 160 can receive data or power from other external electronic device and provide the data or the power to the components of the electronic device 100. The interface 160 can send data from the electronic device 100 to the other external electronic device. For example, the interface 160 can include a wired/wireless headphone port, an external charger port, a wired/wireless data port, a memory card port, an audio input/output port, a video input/output port, and an earphone port.

The camera 170 supports a camera function of the electronic device 100. The camera 170 can capture an object under control of the controller 180 and send the captured data (e.g., an image) to the display 131 and the controller 180. For example, the camera 170 can include a first camera (e.g., a color (RGB) camera) 173 for acquiring color information and a second camera 175 for acquiring iris information.

The first camera (e.g., the color camera) 173 can take a color image of a subject by converting light coming from outside, to an image signal. The first camera 173 can include an image sensor (e.g., a first image sensor) for converting the light to the image signal. The image sensor can adopt a Charged Couple Device (CCD) or a Complementary Metal-Oxide Semiconductor (CMOS). According to an embodiment, the first camera 173 can include a front camera disposed on a front side of the electronic device 100. The front camera can be replaced by the second camera 175, and may not be disposed on the front side of the electronic device 100. The first camera 173 can be disposed on the front side of the electronic device 100 together with the second camera 175. The first camera 173 can include a rear camera disposed on a rear side of the electronic device 100. The first camera 173 can include both of the front camera and the rear camera which are disposed on the front side and the rear side of the electronic device 100 respectively.

The second camera 175 (e.g., an IR camera) can take an iris image of the user using a light source (e.g., IR). The term infrared shall refer to light with a 700 nm-1000 nm wavelength. The second camera 175 can operate as part of a device including the IRIS recognition sensor 195. The second camera 175 can control a focus based on pupil dilation using the IR, process the iris image to a photo, and send the image to the controller 180. The second camera 175 can include an IR generator, and an image sensor which converts the IR reflected from the subject to an image signal. The image signal acquired by the second camera 175 can include depth information (e.g., location information, distance information) of the subject. The IR generator can generate a regular IR pattern in a depth camera which employs an IR structured light scheme, and generates the IR light having general or special profile in a depth camera which employs a Top of Flight (TOF) scheme. Infrared can be used to acquire rich structure of persons with brown eyes which a large percent of the world's population have.

The IR generator can include a light emitter and a light receiver. The light emitter can generate the pattern required to acquire the depth information, for example, IR near-field optical information. The light emitter can project the regular pattern onto a subject to recover in three dimensions. The light receiver can acquire a color image and depth information (e.g., IR information) using the near-field pattern projected by the light emitter. The light receiver can be the first camera 173 or the second camera 175, and acquire the depth information and the color image using one or two cameras. For example, the light receiver can employ a photodiode which detects and converts the incident light to an electric signal. For example, the light receiver can include the photodiode which extracts color information of the light corresponding to a particular visible region of a light spectrum, for example, red light, green light, and blue light.

The image sensor can convert the IR which is emitted by the IR generator to the subject and reflected by the subject, to the image signal. The depth image signal converted from the IR can include distance information from the subject with respect to each IR point to represent different points, for example, pixel values based on the distance from the subject at each IR point of the IR. According to the distance information from the subject, each IR point of the IR can present a relatively small pixel value for a long distance from the subject and represent a relatively large pixel value for a close distance. Like the image sensor (e.g., the first image sensor) of the first camera 173, the image sensor can be implemented using the CCD or the CMOS.

To ease the understanding, the second camera 175 can be referred to as an IR camera in the drawings to be explained. The electronic device 100 can include an iris scanning sensor. The iris scanning sensor can include the IR camera and a light source module. The light source module can output the light. When the IR camera operates, the light source module (e.g., LED) can indicate that the IR camera is operating by turning on/off. The light source module can be disposed near (e.g., in an upper side of the electronic device 100) the IR camera.

The IRIS recognition sensor 195 can analyze and informationize characteristics (e.g., iris shape, iris color, morphology of retina capillary vessels) of the user's iris, and provide corresponding sensing information to the controller 180. For example, the IRIS recognition sensor 195 can code and convert the iris pattern to an image signal, and send the image signal to the controller 180. The controller 180 can compare and determine the iris pattern based on the received image signal. The IRIS recognition sensor 195 can indicate the iris scanning sensor.

The controller 180 can control the operations of the electronic device 100. For example, the controller 180 can perform various controls on user interface displaying, music play, voice communication, data communication, and video communication. The controller 180 can be implemented using one or more processors, or may be referred to as a processor. For example, the controller 180 can include a CP, an AP, an interface (e.g., General Purpose Input/Output (GPIO)), or an internal memory, as separate components or can integrate them on one or more integrated circuits. The AP can conduct various functions for the electronic device 100 by executing various software programs, and the CP can process and control voice communications and data communications. The controller 180 can execute a particular software module (an instruction set) stored in the memory 150 and thus carry out various functions corresponding to the module.

In various embodiments of the present disclosure, the controller 180 can process to detect a display-on event when the display is turned off, to determine whether the display-on event is intended by the user, and to activate the iris scanning sensor (e.g., the IRIS recognition sensor 195) when the display-on event is intended by the user. A display-on event is an event that causes a display that is turned off (de-illuminated) to be turned on (illuminated). The controlling of the controller 180 according to various embodiments of the present disclosure is now described with the drawings.

The controller 180 according to an embodiment of the present disclosure can control the above-stated functions and various operations of typical functions of the electronic device 100. For example, the controller 180 can control particular application execution and a screen display. The controller 180 can receive an input signal corresponding to various touch event or proximity event inputs supported by a touch-based or proximity-based input interface (e.g., the touch screen 130), and control its function. Also, the controller 180 may control various data transmissions/receptions based on the wired communication or the wireless communication.

The power supply 190 can receive external power or internal power and supply the power required to operate the components under control of the controller 180. The power supply 190 can supply or cut the power to display 131 and the camera 170 under the control of the controller 180.

In some cases, various embodiments of the present disclosure can be implemented by the controller 180. According to software implementation, the procedures and the functions in embodiments of the present disclosure may be implemented by separate software modules. The software modules can execute one or more functions and operations described in the specification.

According to various embodiments, an electronic device can include a memory 150, a display 131, an iris scanning sensor (e.g., an IRIS recognition sensor 195), and a processor (e.g., the controller 180) functionally coupled with at least one of the memory, the display, and the iris scanning sensor, wherein the processor detects a display-on event, determines whether the display-on event is an intended input of a user, and activates the iris scanning sensor when the display-on event is the intended user input.

For the intended user input, the processor can display a first user interface which guides iris recognition, on the display. Intended user input is intentional input to the electronic device by the user with the intent or knowledge that a display screen event will occur and can include at least one of button selection, touch input detection, and cover case opening in the electronic device.

The iris scanning sensor can include an IR camera or a light source module, and the processor operates the IR camera or the light source module while displaying the first user interface. It shall be understood that activation of an IR camera and activation of a camera shall include operation of the camera where the camera receives the image and is capable of immediately capturing the image, such as in response to selection of a virtual button by a user, or a control signal or command to capture from the controller 180, and shall be understood to include not just the time period that the camera captures the image. Deactivation of the IR camera and deactivation of a camera shall be understood to mean that the camera is not inputting the image from the lens into the electronic device. For the intended user input, the processor can perform iris recognition by activating the iris scanning sensor, and when completing the iris recognition, display a user interface for unlocking based on the input.

For the intended user input, the processor can perform iris recognition by activating the iris scanning sensor and display a user interface for user authentication on the display during the iris recognition.

The user interface for the user authentication can include a first interface for iris-based user authentication and another interface of at least one of pattern-based user authentication, password-based user authentication, and fingerprint-based user authentication.

The processor can conduct other user authentication during the iris-based user authentication.

For an unintended user input, the processor can display a second user interface which does not guide iris recognition. While displaying the second user interface, the processor can refrain from activating the IR camera or the light source module. When detecting a user touch input in the second user interface, the processor can perform iris recognition by activating the iris scanning sensor.

The unintended user input can include inputs to the electronic device that are not user inputs, such as receiving at least one of incoming push message (text, phone call), the result of user inputs that are not contemporaneous with the display-on events, such as an alarm call in the electronic device, and can include user inputs that are not for the purpose of turning on the display such as a charger connection, and external device connection.

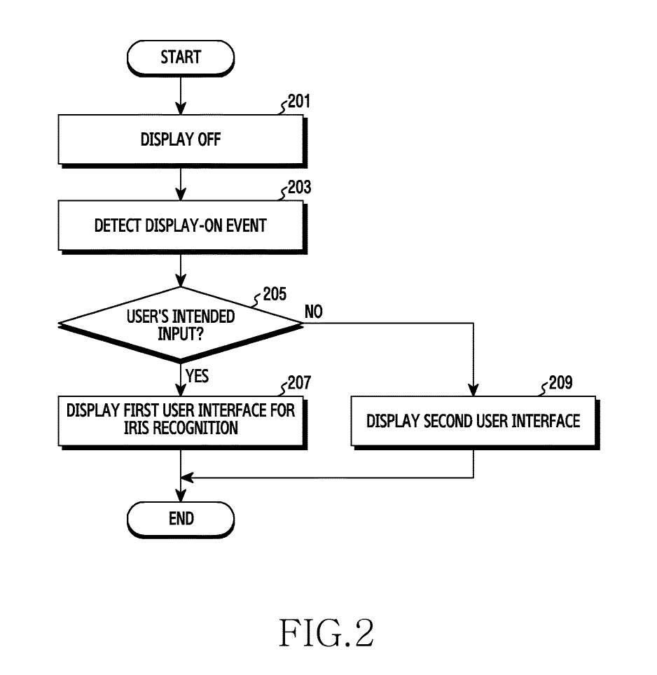

FIG. 2 is a flowchart of a method according to various embodiments.

Referring to FIG. 2, in operation 201, the electronic device (e.g., the controller 180) can turn off a display. The display-off state can indicate that the display 131 of the electronic device 100 is turned off. After turning on the display 131 and receiving no user input during a preset time, the controller 180 can turn off the display 131. Alternatively, when receiving a display-off command (e.g., selecting a power button (or a lock button)) from the user, the controller 180 can turn off the display 131.

In operation 203, the electronic device (e.g., the controller 180) can detect a display-on event. The display-on event can indicate that the display 131 of the electronic device 100 is turned on, i.e., illuminated when it was previously de-illuminated. Display-on events can include, but are not limited to, user selection of a power button, home button, preset touch input, tapping, opening or closing of the cover, receipt of a push message from a base station, connection to a charger, alarm, and low-battery level to name a few.

For example, when the user selects a power button or a home button, the controller 180 can determine the display-on event. Alternatively, when detecting a preset touch input (e.g., a gesture tapping on the display 131) from the user, the controller 180 can determine the display-on event. For example, the gesture tapping on the display 131 can tap on the display 131 more than twice in succession. When the electronic device is covered with a cover case, the controller 180 can turn on/off the display 131 as the cover case is opened/closed. When the cover case is open, the controller 180 can determine the display-on event.

Alternatively, when receiving a push message from a server or a base station, the controller 180 can determine the display-on event. When the electronic device is connected to a charger, the controller 180 can determine the display-on event. When the electronic device 100 is connected to an external device (e.g., an earphone), the controller 180 can determine the display-on event.

When a preset alarm sounds in the electronic device 100, the controller 180 can determine the display-on event. For example, when a current time is the alarm time which is set by the user, an alarm application can sound the alarm. Alternatively, when a current date (or time) is a date which is set by the user, a calendar application can sound the alarm. Alternatively, when a battery of the electronic device 100 falls below a preset level (e.g., 20%), the alarm can notify the low battery level. Besides, the alarm can include various alarms (or notifications) set in the electronic device 100.

In operation 205, the electronic device (e.g., the controller 180) can determine whether the display-on event is intended by the user. The input intended by the user can indicate that the user originates the display-on event. For example, when the power button (or the home button) is pressed, a preset touch input is detected, or the cover case is open in the display-off state, the controller 180 can determine the user's intended input. In the display-off state, it is less likely that the power button is pressed, the touch (e.g., a gesture for tapping on the display 131) is input, or the cover case is opened without user's intervention. Accordingly, upon detecting such operations, the controller 180 can determine the user's intended input.

In the display-off state, when a push message is received, a preset alarm of the electronic device 100 sounds, a charger is connected, or an external device is connected, the controller 180 can determine no user's intended input. In the display-off state, when the push message is received, the alarm sounds, the charger is connected, or the external device is connected, the controller 180 can be configured to automatically turn on the display 131. That is, upon receiving the push message, the controller 180 can automatically turn on the display 131 and notify the incoming push message to the user. When the preset alarm of the electronic device 100 sounds, the controller 180 can automatically turn on the display 131 and notify alarm contents (e.g., alarm time (date), internal alarm) to the user. When the charger is connected, the controller 180 can automatically turn on the display 131 and notify the charger connection to the user. When the external device is connected, the controller 180 can automatically turn on the display 131 and notify the external device connection to the user. Thus, the controller 180 detecting such operations can determine the user's unintended input.

Therefore, in certain embodiments, the controller 180 can determine whether the display-on event is user intended or user unintended based on the following chart:

| | |
|---|---|
| User Intended | User Unintended |
| Power button | Receipt of a Push Message |
| Touching of the | Connection of the Charger |
| touchscreen | |
| Opening the Case | Alarm (clock, battery level, other condition) |
| | Connection by another device, including |
| | pairing |

When determining that the display-on event is the user's intended input in operation 205, the controller 180 can proceed to operation 207. When determining that the display-on event is a user's unintended input in operation 205, the controller 180 can proceed to operation 209.

In operation 207, the electronic device (e.g., the controller 180) can display a first user interface regarding the iris recognition. The first user interface can include a guide message (e.g., Look here) notifying the iris recognition. In response to the display-on event, the controller 180 can apply the power to the display 131. In this case, the display 131 can be turned on. When the power is applied to the display 131, the display 131 can display the first user interface under control of the controller 180.

For example, when the display 131 is turned off, the electronic device 100 may be locked. When the display 131 is turned on, the controller 180 can display a lock screen of the electronic device 100. The lock screen can be a screen requiring user authentication (e.g., password entering, pattern input, fingerprint scanning, iris scanning). For example, the lock screen can show a background image (e.g., a lock notification screen or an image selected by the user) of the electronic device 100. For example, when receiving a push message, the lock screen can display notification (e.g., a message popup) of the incoming push message. The first user interface can display an iris recognition guide message or an incoming push message notification on the background image.

The controller 180 can automatically activate and operate an IR camera (e.g., the second camera 175) for the iris recognition in operation 207. Also, the controller 180 can turn on/off a light source module (e.g., LED) for the iris recognition in operation 207. That is, while displaying the first user interface on the display 131, the controller 180 can activate the IR camera or the light source module.

In operation 209, the electronic device (e.g., the controller 180) can display a second user interface. The second user interface can be distinguished from the first user interface. The second user interface can include a guide message (e.g., Tap here . . . ) notifying no execution of the iris recognition. In response to the display-on event, the controller 180 can apply the power to the display 131. In this case, the display 131 can be turned on. When the power is applied to the display 131, the display 131 can display the second user interface under control of the controller 180.

Similarly to the first user interface, the second user interface can be related to the lock screen. For example, the lock screen can display the background image of the electronic device 100. For example, when receiving a push message, the lock screen can display a notification (e.g., a message popup) of the incoming push message. The second user interface can display a guide message to enable iris recognition or the incoming push message notification on the background image.

In certain embodiments, the second user interface may differ from the first interface by refraining from activating and operating the IR camera for the iris recognition in operation 209. That is, by not applying the power to the IR camera, the controller 180 can refrain from activating the IR camera. Also, the controller 180 may not operate the light source module for the iris recognition in operation 209. That is, by not applying the power to the light source module, the controller 180 can control not to activate the light source module. While displaying the second user interface on the display 131, the controller 180 can deactivate the IR camera or the light source module. In certain embodiments, the controller 180 can activate the IR camera in response to and following a user input following a prompt.

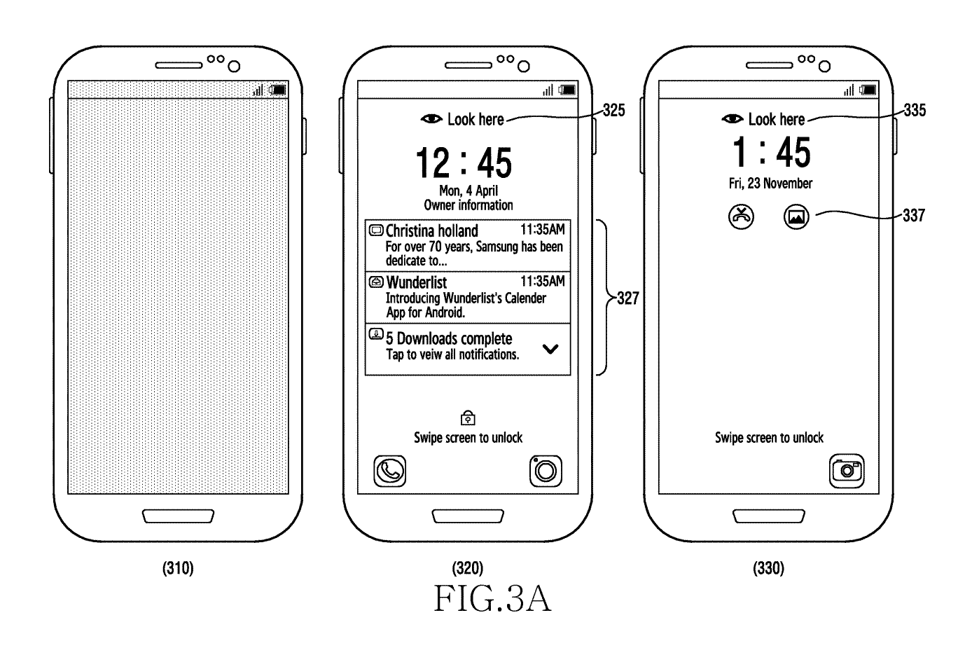

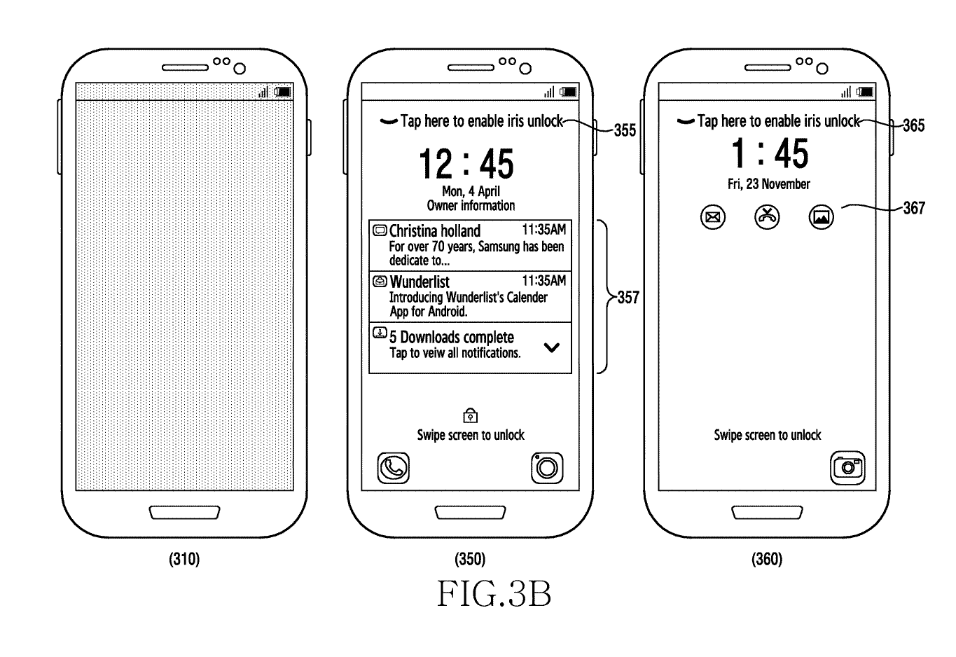

FIGS. 3A and 3B depict a user interface for iris recognition according to various embodiments.

FIG. 3A depicts a user interface for the iris recognition.

Referring to FIG. 3A, a first user interface 310 can be displayed when the display 131 of the electronic device 100 is turned off. In the display-off state, the controller 180 can display the first user interface 310. The controller 180 can detect a display-on event in the display-off state. When the display-on event corresponds to a user's intended input (e.g., lock button selection, touch input, cover case open), the controller 180 can display user interface 320. The user interface 320 can include a guide message 325 for commencing iris recognition, current date and time (e.g., 12:45, Mon, 4 April), and an incoming push message notification 327. While displaying the second user interface 320 on the display 131, the controller 180 can activate and operate the IR camera or the light source module. The second user interface 320 can display a text message notifying “iris recognition” at the bottom.

The guide message 325 for the iris recognition can include a guide image (e.g., an open eye image) for the iris recognition, a guide text (e.g., Look here), a video (e.g., a moving icon), or their combination. The guide message 325 for the iris recognition can guide the user to look at the top end of the electronic device 100 in relation with a mounting position of the IR camera. Also, the guide message 325 for the iris recognition can notify that the electronic device 100 performs the iris recognition. The incoming push message notification 327 can provide a popup window of a list including push messages received or unviewed during a certain period. The incoming push message notification 327 may not be displayed when no push message is received. For example, the incoming push message notification 327 can include at least one of a sender (e.g., Christina holland, Wunderlist), a reception time (e.g., 11:35 AM), message contents (e.g., For over 70 years, Introducing Wunderlist's), and alarm details (e.g., 5 Downloads complete . . . ) of each push message.

When the display-on event corresponds to the user's intended input, the controller 180 can display user interface 330. The user interface 330 can include a guide message 335 for the iris recognition, current date and time (e.g., 1:45, Fri, 23 November), and an incoming push message notification 337. The guide message 335 for the iris recognition can include a guide image (e.g., an open eye image) for the iris recognition, a guide text (e.g., Look here), a video (e.g., a moving icon), or their combination. The guide message 335 for the iris recognition can be the same as or similar to the iris recognition guide message 325 of the second user interface 320. While displaying the third user interface 330 on the display 131, the controller 180 can operate the IR camera or the light source module.

The incoming push message notification 337 can provide icons of push messages received or unviewed for a certain period. For example, the incoming push message notification 337 can display an icon based on attributes of an application relating to the push message. For example, when the push message is related to a call application, the controller 180 can generate an icon (e.g., a phone shape) regarding the call application, as the incoming push message notification 337. When the push message is related to a gallery application, the controller 180 can generate an icon (e.g., a photo shape) regarding the gallery application, as the incoming push message notification 337.

When the display-on event corresponds to the user's intended input, the controller 180 can display the second user interface 320 or the third user interface 330. Alternatively, when the second user interface 320 is displayed and then a certain time (e.g., 3 seconds, 5 seconds, etc.) elapses, the controller 180 can display the third user interface 330. For example, the controller 180 can generate an icon of the push message based on the push message of the second user interface 320, and display the third user interface 330 which provides the incoming push message notification 337 as the generated icon.

FIG. 3B depicts a second user interface with the IR camera deactivated.

Referring to FIG. 3B, the controller 180 can display user interface 310 in the display-off state. The controller 180 can detect a display-on event in the display-off state. When the display-on event corresponds to a user's unintended input (e.g., incoming push message, alarm call, charger connection, external device connection), the controller 180 can display user interface 350. The user interface 350 can include a guide message 355 to enable the iris recognition, current date and time (e.g., 12:45, Mon, 4 April), and an incoming push message notification 357. While displaying the user interface 350 on the display 131, the controller 180 does not activate or operate the IR camera or the light source module. The user interface 350 can display a text message notifying “to enable iris recognition” at the bottom.

The guide message 355 to enable the iris recognition can include a guide image (e.g., a closed eye image) notifying the iris recognition is not conducted, a guide text (e.g., Tap here to enable iris unlock), a video (e.g., a moving icon), or their combination. The guide message 355 to enable the iris recognition can guide to a separate user input required for the iris recognition. Also, the guide message 355 to enable the iris recognition can notify that the electronic device 100 does not conduct the iris recognition. The incoming push message notification 357 can provide a list of push messages received or unviewed during a certain time. For example, the incoming push message notification 357 can include at least one of a sender (e.g., Christina holland, Wunderlist), a reception time (e.g., 11:35 AM), message contents (e.g., For over 70 years, Introducing Wunderlist's), and alarm details (e.g., 5 Downloads complete . . . ) of each push message.

When the display-on event corresponds to the user's unintended input, the controller 180 can display user interface 360. The user interface 360 can include a guide message 365 to enable the iris recognition, current date and time (e.g., 1:45, Fri, 23 November), and an incoming push message notification 367. The guide message 365 to enable the iris recognition can include a guide image (e.g., a closed eye image) notifying that the iris recognition is not conducted, a guide text (e.g., Tap here to enable iris unlock), a video (e.g., a moving icon), or their combination. The guide message 365 to enable the iris recognition can be the same as or similar to the guide message 355 to enable the iris recognition of the fourth user interface 350. As displaying the fifth user interface 360 on the display 131, the controller 180 does not operate or activate the IR camera or the light source module.

The incoming push message notification 367 can provide icons of push messages received or unviewed for a certain time. For example, the incoming push message notification 367 can display an icon based on attributes of an application relating to the push message. For example, when the push message is related to a message application, the controller 180 can generate an icon (e.g., a letter shape) regarding the message application, as the incoming push message notification 367.

When the display-on event corresponds to the user's unintended input, the controller 180 can display user interface 350 or user interface 360. Alternatively, when the fourth user interface 350 is displayed and then a certain time elapses, the controller 180 can display the fifth user interface 360. For example, the controller 180 can generate a push message icon based on the push message of the fourth user interface 350, and display the fifth user interface 360 which provides the incoming push message notification 367 as the generated icon.

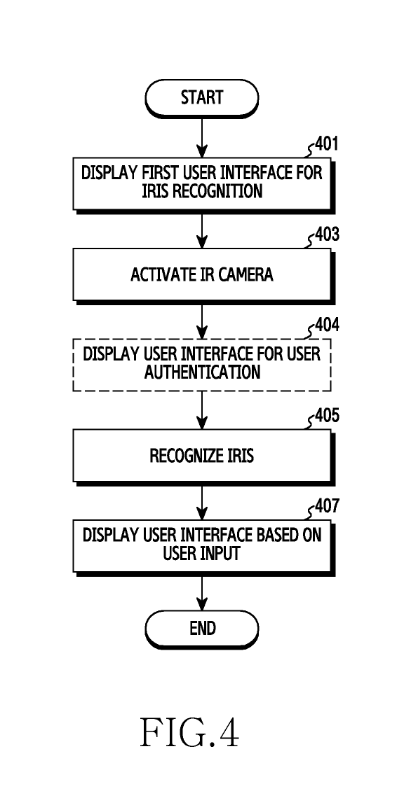

FIG. 4 is a flowchart of an iris recognition method of an electronic device according to various embodiments. FIG. 4 illustrates a detailed method for recognizing an iris according to a user's intended input.

Referring to FIG. 4, in operation 401, the electronic device (e.g., the controller 180) can display a first user interface regarding the iris recognition. The first user interface can include a guide message notifying the iris recognition. The first user interface has been described in FIG. 3A and thus shall not be further explained.

In operation 403, the electronic device (e.g., the controller 180) can activate an IR camera. Activating the IR camera can mean that the power is applied to the IR camera to operate the IR camera. The controller 180 can activate the IR camera while displaying the first user interface on the display 131. The controller 180 may activate a light source module. The controller 180 can control the light source module to turn on/off and thus notify the iris recognition to the user. While displaying the first user interface on the display 131, the controller 180 can activate the IR camera or the light source module.

In operation 404, the electronic device (e.g., the controller 180) can display a user interface for user authentication. When the display 131 is turned off, the electronic device 100 is locked. When the display 131 is turned on, the controller 180 can display a lock screen of the electronic device 100. The user interface for the user authentication can notify that the user authentication is required to unlock the electronic device 100. The controller 180 may or may not display the user interface for user authentication during the iris recognition. That is, the controller 180 may conduct operation 404 in between operation 403 and operation 405, or may not conduct operation 404.

In operation 405, the electronic device (e.g., the controller 180) can perform the iris recognition. The controller 180 can activate the IR camera and capture a user's eye using the IR camera. The iris can be recognized during a predetermined time (e.g., 10 seconds, 15 seconds, etc.). According to an embodiment, the controller 180 can abort the iris recognition when the iris recognition is executed normally within the preset time, and perform a related operation (e.g., unlock). The iris authentication can determine whether an iris image currently acquired in operation 405 matches an iris image stored in the memory 150 of the electronic device 100. When the iris is authenticated (e.g., when the current iris image matches the stored iris image), the controller 180 can proceed to operation 407.

When the iris is not authenticated normally during the predetermined time, the controller 180 can abort the iris recognition and output a guide on the display 131. When the iris is not authenticated normally during the preset time, the controller 180 can display a guide message for the iris recognition and re-perform the iris recognition. Alternatively, when the iris is not authenticated (e.g., when the current iris image does not match the stored iris image), the controller 180 can display a guide message for the iris recognition and re-perform the iris recognition. When the iris is not authenticated (e.g., when the current iris image does not match the stored iris image), the controller 180 may request other user authentication (e.g., password input, pattern input, fingerprint scanning) than the iris recognition.

In operation 407, the electronic device (e.g., the controller 180) can display a user interface based on a user input. For example, when the iris is successfully authenticated, the controller 180 can display the user interface based on the user input. When displaying the first user interface according to lock button (or the home button) selection or preset touch input, the controller 180 can display a user interface (e.g., a home screen, an application execution screen) before the locking or an unlocked user interface. In the unlocked user interface, the user can use functions of the electronic device that are generally restricted in the locked states, such as making phone calls (to non-emergency numbers), texting, emailing, and using payment applications to make payments. Alternatively, when any one push message is selected (tapped) in the first user interface, the controller 180 can display an application execution screen corresponding to the push message. For example, the controller 180 can display a main screen or a reply screen of the message application as an execution screen of the message application based on the user input.

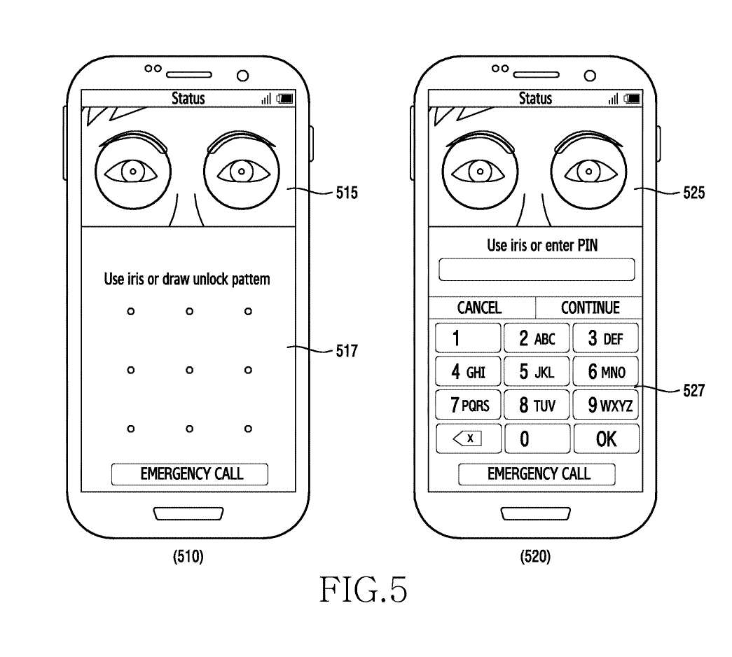

FIG. 5 depicts a user interface for user authentication according to various embodiments.

Referring to FIG. 5, the controller 180 can display a user interface 510 for first user authentication and a user interface 520 for second user authentication, including a first interface for iris-based user authentication and another interface (e.g., a second interface, a third interface, etc.) for another user authentication (e.g., pattern-based user authentication, password-based user authentication, fingerprint-based user interface, etc.). The electronic device 100 can provide two or more user authentication types together, and process one authentication or complex authentications.

The user interface 510 for the first user authentication can include a first interface 515 for the iris-based user authentication and a second interface 517 for the pattern-based user authentication. The first interface 515 can include a text (e.g., “Use iris or draw unlock pattern”) for intuitively guiding the user's iris authentication (e.g., a gaze direction), graphics (e.g., an image or an icon corresponding to the user's eye), or their combination. The graphics may be provided based on a preview image captured by a front camera (e.g., the first camera 173) or an IR camera (e.g., the second camera 175) of the electronic device 100. The second interface 517 can include a text (e.g., Use iris or draw unlock pattern) for guiding to input a pattern for the user's pattern authentication, graphics (e.g., a 3×3 pattern input field), or their combination.

The user interface 520 for the second user authentication can include a first interface 525 for the iris-based user interface, and a third interface 527 (e.g., an input field, a keypad) for the password-based user authentication. The first interface 525 can include a text for intuitively guiding the user's iris authentication (e.g., a gaze direction), graphics (e.g., an image or an icon corresponding to the user's eye), or their combination. The third interface 527 can include a keypad (or a keyboard) for entering a password, and an input field for displaying the password entered by the user through the keypad. The input field can show the entered password (e.g., show symbols (*, #)) which is secured.

While displaying the user interface for the user authentication, the electronic device 100 can recognize the iris by activating the IR camera. The iris recognition can terminate according to user's proximity, or automatically terminate after a preset time (e.g., 10 seconds, 15 seconds, 20 seconds, etc.). During the iris-based user authentication or after the iris recognition, the user may conduct the pattern-based user authentication using the second interface 517 or the password-based user authentication using the third interface 527. During the iris recognition, the electronic device 100 can process user authentication (e.g., password or pattern authentication) in parallel or in sequence. After the iris recognition ends, the electronic device 100 can process the user authentication (e.g., password- or pattern-based authentication) according to a user manipulation.

The electronic device 100 can simultaneously process one or more user authentication types. According to an embodiment, the electronic device 100 can process the iris-based authentication based on an iris image acquired by the iris recognition and a reference iris image (e.g., an iris image stored in the memory 150), and independently and concurrently process the pattern-based authentication based on the pattern entered by the user and a reference pattern. According to an embodiment, the electronic device 100 can process the iris-based authentication based on an iris image acquired by the iris recognition and the reference iris image (e.g., an iris stored in the memory 150), and independently and concurrently process the password-based authentication based on the password entered by the user and a reference password (e.g., a password stored in the memory 150).

As displaying the user interface for the user authentication, the electronic device 100 may provide fingerprint-based user authentication using the home button. For example, the electronic device 100 can include a fingerprint scanning sensor inside the home button, and the user can conduct the fingerprint-based user authentication by touching or rubbing his/her fingerprint to the home button. The electronic device 100 may recognize the iris for the iris-based user authentication even during the user's fingerprint-based authentication. For example, the electronic device 100 can provide two or more user authentication types together, and process the authentication types in sequence or in combination.

In the lock state of the electronic device 100, the user interface for the user authentication can be displayed in a screen switch manner in response to a user's input (e.g., swipe). The user interface for the user authentication may be provided in response to the user input (e.g., object or menu selection for the user authentication, an operation (e.g., logon, financial transaction, digital commerce) requiring the user authentication) while executing a particular application.

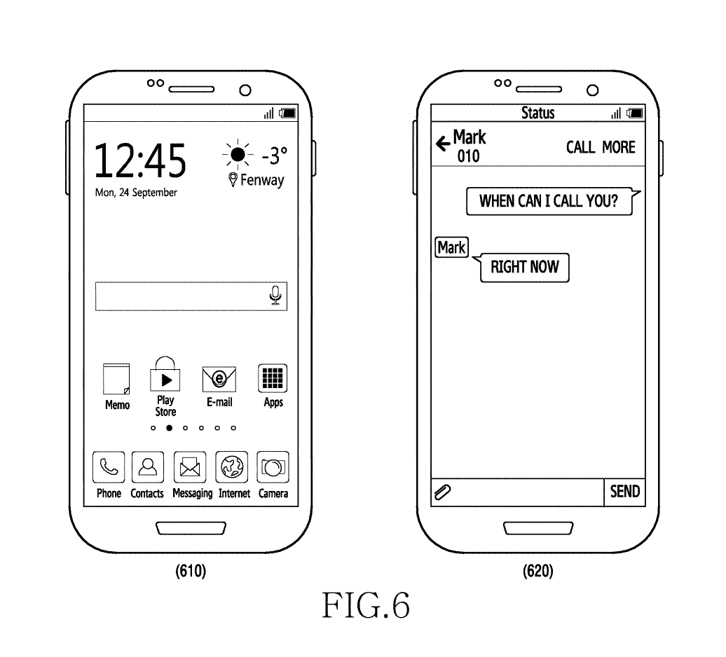

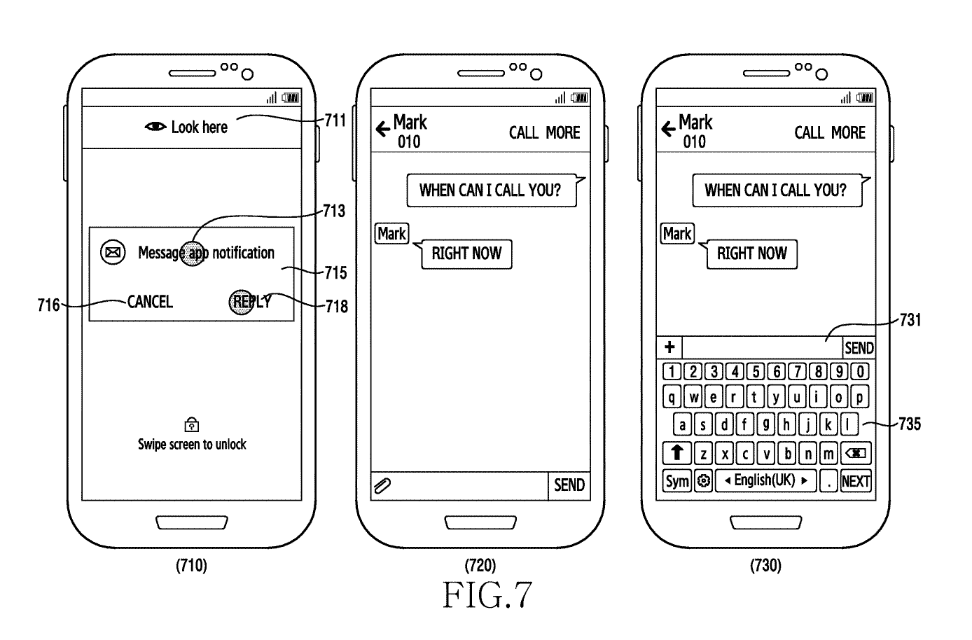

FIGS. 6 and 7 are diagrams of a user interface based on a user input according to various embodiments (such as during step 407).

FIG. 6 depicts a user interface after user authentication is completed while a first user interface for iris recognition is displayed.

Referring to FIG. 6, when the user authentication (e.g., iris, password, pattern, fingerprint) is permitted, the electronic device (e.g., the controller 180) can display a user interface 610 or 620 to unlock electronic device. For example, when conducting the user authentication while displaying the first user interface regarding the iris recognition according to the lock button (or the home button) selection or a preset touch input, the controller 180 can display the first user interface 610 of the home screen or the second user interface 620 of an application execution screen. The first user interface 610 can be a home screen showing images (e.g., icons) corresponding to one or more applications installed on the electronic device 100. Alternatively, the second user interface 620 can be a screen of a message application executed.

That is, the first user interface 610 or the second user interface 620 can be the user interface (e.g., the user interface before locking) before the display 131 of the electronic device 100 is turned off

FIG. 7 depicts a user interface after user authentication is completed according to a user's touch input detected while a first user interface for iris recognition is displayed.

Referring to FIG. 7, when detecting a user's touch input in a first user interface for iris recognition during the user authentication, the electronic device (e.g., the controller 180) can display a second user interface 710 based on the touch input. For example, the second user interface 710 can be displayed when the user selects (e.g., taps) on any one push message in the first user interface. The second user interface 710 can include a guide message 711 for the iris recognition, and a message 715 for notifying that the push message selected by the user is an incoming message. The incoming message notification message 715 can include an item (or button) 713 for viewing the incoming message, an item 718 for replying to the incoming message, and a CANCEL item 716.

When the VIEW item 713 is selected in the incoming message notification message 715, after the user authentication (e.g., iris, password, pattern, fingerprint) is permitted, the controller 180 can display a third user interface 720. The third user interface 720, which is to view the incoming message, can be an execution screen of a message application. The third user interface 720 can display contents of the incoming message. When a REPLY item 718 is selected in the incoming message notification message 715, after the user authentication is permitted, the controller 180 can display a fourth user interface 730. The fourth user interface 730, which is to send a reply to the incoming message, can be a reply screen of the message application. The fourth user interface 730 can include an input field 731 and a keypad 735 for sending the reply to the message.

Hence, the controller 180 can display a different unlock screen according to the user's selection in the first user interface for the iris recognition.

After displaying the second user interface 710, the controller 180 can display the user interface 610 or 620 of FIG. 6 for the user authentication before displaying the third user interface 720 or the fourth user interface 730.

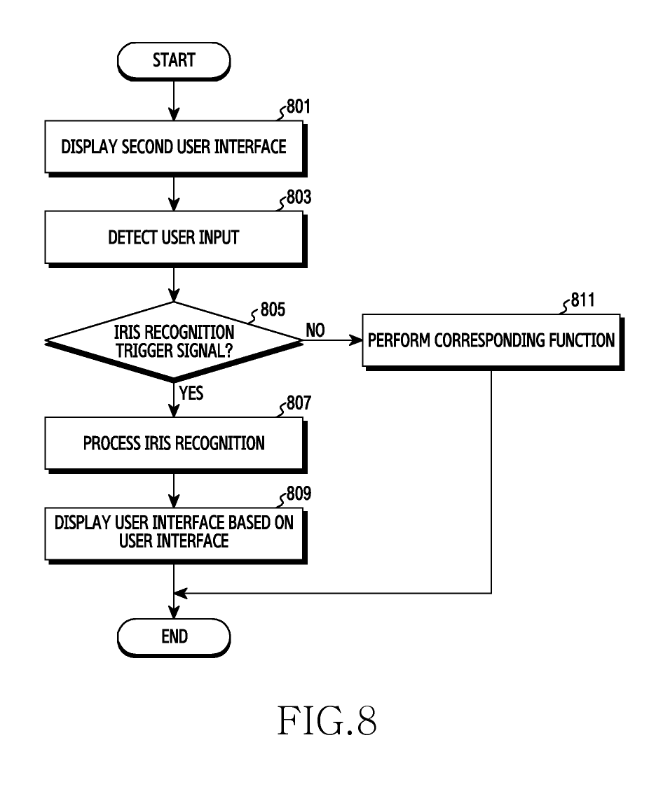

FIG. 8 is a flowchart of an iris recognition method of an electronic device according to various embodiments.

Referring to FIG. 8, in operation 801, the electronic device (e.g., the controller 180) can display a second user interface. The second user interface can include a guide message notifying that the iris recognition is not conducted. The second user interface can be one of the interfaces shown FIG. 3B.

In operation 803, the electronic device (e.g., the controller 180) can detect a user input. The controller 180 can detect the user input while displaying the second user interface. For example, the user input can include lock button (or home button) selection, volume control key selection, and touch input. Such a user input may or may not be a trigger signal for the iris recognition.

In operation 805, the electronic device (e.g., the controller 180) can determine whether the user input is the iris recognition trigger signal. For example, when a guide message to enable iris recognition is selected or an incoming push message notification is selected in the second user interface, the controller 180 can determine the user input as the trigger signal for the iris recognition. Alternatively, when an item not requiring the user authentication is selected in the second user interface, the controller 180 can determine that the user input is not the trigger signal for the iris recognition.

When the user input is the trigger signal for the iris recognition in operation 805, the controller 180 can perform operation 807. When the user input is not the trigger signal for the iris recognition in operation 805, the controller 180 can perform operation 811.

In operation 807, the electronic device (e.g., the controller 180) can activate the IR camera and process the iris recognition. For example, the iris recognition can include the operations of FIG. 4. For example, when the user input is the trigger signal for the iris recognition, the controller 180 can display a first user interface for the iris recognition. The first user interface can be modified from the second user interface by changing the guide message to enable iris recognition (e.g., the message to enable iris recognition 355 of FIG. 3B) to the iris recognition guide message (e.g., the iris recognition guide message 325 of FIG. 3A). The controller 180 can activate an IR camera, display a user interface for user authentication, and carry out the iris recognition.

The iris recognition can include operations 403 and 405 of FIG. 4. The controller 180 can modify the guide message to enable iris recognition (e.g., the message to enable iris recognition 355 of FIG. 3B) to the iris recognition guide message (e.g., the iris recognition guide message 325 of FIG. 3A) in the second user interface, activate the IR camera, display the user interface for the user authentication, and carry out the iris recognition.

In operation 809, the electronic device (e.g., the controller 180) can display a user interface based on a user input. For example, when the iris authentication is successful, the controller 180 can display the user interface based on the user input. When detecting a user input which selects the guide message to enable iris recognition in the second user interface, the controller 180 can display a user interface (e.g., a home screen, an application execution screen) before the locking. Alternatively, when the user selects (taps) on one push message in the second user interface, the controller 180 can display an application execution screen corresponding to the push message. The application execution screen can be a push message view screen or a push message reply screen. Operation 809 can be the same as or similar to operation 407 of FIG. 4.

In operation 811, the electronic device (e.g., the controller 180) can perform a function corresponding to the user input. For example, when a lock button (or the home button) is selected in the second user interface, the contro11er180 can turn off the display 131. Alternatively, when a volume control key is selected in the second user interface, the controller 180 may or may not control sound volume. In the lock state, the sound volume may or may not be controlled according to setting of the electronic device 100. When the electronic device 100 is configured to control the sound volume in the lock state, it can control the sound according to the volume control key. When the electronic device 100 is not configured to control the sound volume in the lock state, it can disregard the selection of the volume control key. Alternatively, when the electronic device 100 is not configured to control the sound volume in the lock state, it may display a popup message guiding to unlock for the volume control. Alternatively, when emergency call is selected in the second user interface, the controller 180 may provide a keypad for the emergency call.

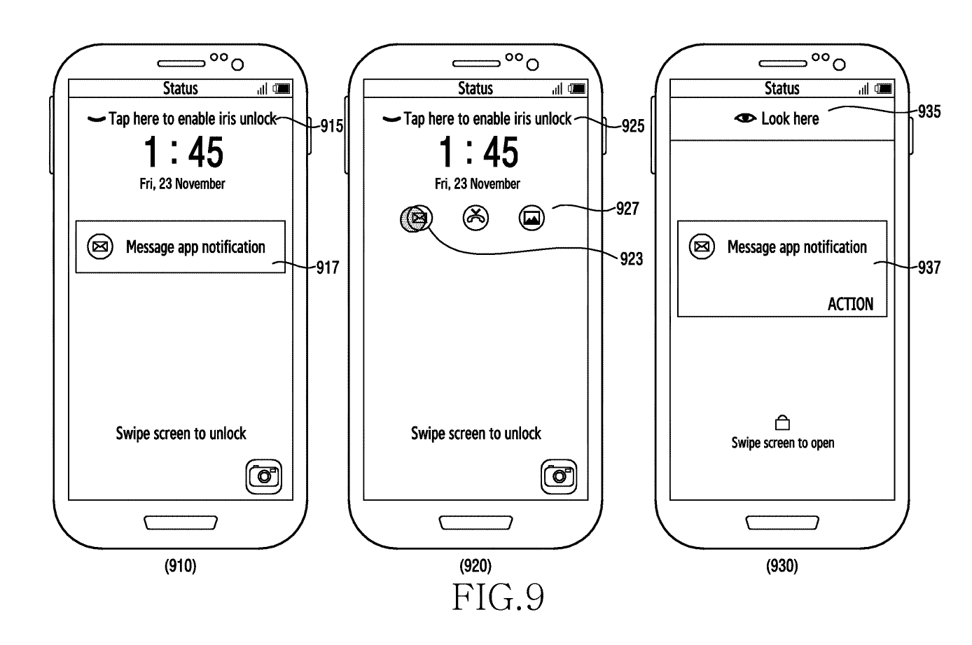

FIG. 9 depicts another user interface based on a user intention according to various embodiments.

Referring to FIG. 9, when a display-on event corresponds to a user's unintended input (e.g., incoming push message, alarm call, charger connection, external device connection), the controller 180 can display a first user interface 910. The first user interface 910 can include a guide message 915 to enable the iris recognition, current date and time (e.g., 1:45, Fri, 23 November), and an incoming push message notification 917. The incoming push message notification 917 can show an incoming push message as a popup. As displaying the first interface 910 on the display 131, the controller 180 does activate or operate an IR camera or a light source module.

When the display-on event corresponds to the user's unintended input, the controller 180 can display a second user interface 920. The second user interface 920 can include a guide message 925 to enable the iris recognition, current date and time (e.g., 1:45, Fri, 23 November), and an incoming push message notification 927. The incoming push message notification 927 can show an icon of the push message. As displaying the second user interface 920 on the display 131, the controller 180 does not activate or operate the IR camera or the light source module.

The controller 180 can detect a user input in the first user interface 910 or the second user interface 920, and provide a third user interface 930 when the detected user input is an iris recognition trigger signal. For example, when the incoming push message notification 917 of the first user interface 910 is selected (tapped), the controller 180 can display the third user interface 930. When a message icon 923 of the incoming push message notification 917 is selected (tapped) in the second user interface 920, the controller 180 can display the third user interface 930. The third user interface 930 can include a guide message 937 for the iris recognition and a guide message 937 notifying that the push message selected by the user is an incoming message. The third user interface 930 can be the same as or similar to the second user interface 910 of FIG. 7.

The incoming message notification message 937 can include an item (e.g., Message app notification) for viewing the incoming message, and an item (e.g., action) for replying to the incoming message. When the view item is selected in the incoming message notification message 937, the controller 180 can display the third user interface 720 of FIG. 7. When the reply item is selected in the incoming message notification message 937, the controller 180 can display the fourth user interface 730 of FIG. 7. After displaying the third user interface 930, the controller 180 can display the user interface 610 or 620 of FIG. 6 for the user authentication before displaying the third user interface 720 or the fourth user interface 730.

According to various embodiments, a method for operating an electronic device which includes an iris scanning sensor can include detecting a display-on event in a display-off state, determining whether the display-on event is an intended user input, and when the display-on event is the user intended input, activating the iris scanning sensor.

Activating the iris scanning sensor can include, for the intended user input, displaying a first user interface which guides iris recognition.

The iris scanning sensor can include an IR camera or a light source module, and activating the iris scanning sensor can further include, when displaying the first user interface, determining to activate the IR camera or the light source module.

The method can further include, for an unintended user input, displaying a second user interface which guides no iris recognition.

The method can further include, when displaying the second user interface, determining to deactivate the IR camera or the light source module.

The method can further include, when detecting a user touch input in the second user interface, performing iris recognition by activating the iris scanning sensor.

The intended user input can include at least one of button selection, touch input detection, and cover case opening in the electronic device.

The unintended user input can include at least one of incoming push message, alarm call in the electronic device, charger connection, and external device connection.

Various embodiments of the present disclosure can be implemented in a recording medium which can be read by a computer or a similar device using software, hardware or a combination thereof. According to hardware implementation, various embodiments of the present disclosure can be implemented using at least one of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, and electric units for performing other functions.

According to an embodiment of the present disclosure, the recording medium can include a computer-readable recording medium which records a program for detecting a display-on event in a display-off state, determining whether the display-on event is an intended user input, and, when the display-on event is the user intended input, activating the iris scanning sensor.

While the invention has been shown and described with reference to certain exemplary embodiments thereof, it will be understood by those skilled in the art that various changes in form and details may be made therein without departing from the spirit and scope of the invention as defined by the appended claims and their equivalents.

What is claimed is:

1. An electronic device comprising:
a display;
an iris scanning sensor; and
a processor functionally coupled with the display, and the iris scanning sensor,
wherein the processor activates the iris scanning sensor when receiving a display-on event in a display-off state that is an intended user input.

2. The electronic device of claim 1, wherein, for the intended user input, the processor displays a first user interface which guides iris recognition, on the display.

3. The electronic device of claim 2, wherein the iris scanning sensor comprises:
an Infrared (IR) camera and a light source, and
while displaying the first user interface, the processor operates the IR camera and the light source.

4. The electronic device of claim 1, wherein, for an unintended user input, the processor displays a second user interface which guides no iris recognition, on the display.

5. The electronic device of claim 4, wherein the iris scanning sensor comprises:
an IR camera or a light source module, and
while displaying the second user interface, the processor deactivates the IR camera or the light source module.

6. The electronic device of claim 4, wherein, when detecting a user touch input in the second user interface, the processor performs iris recognition by activating the iris scanning sensor.

7. The electronic device of claim 1, wherein the intended user input comprises at least one of button selection, touch input detection, and cover case opening in the electronic device.

8. The electronic device of claim 1, wherein the unintended user input comprises at least one of incoming push message, alarm call in the electronic device, charger connection, and external device connection.

9. The electronic device of claim 1, wherein, for the intended user input, the processor performs iris recognition by activating the iris scanning sensor, and when completing the iris recognition, displays a user interface for unlocking based on the input.

10. The electronic device of claim 1, wherein, for the intended user input, the processor performs iris recognition by activating the iris scanning sensor and displays a user interface for user authentication on the display during the iris recognition.

11. The electronic device of claim 10, wherein the user interface for the user authentication comprises a first interface for iris-based user authentication and another interface of at least one of pattern-based user authentication, password-based user authentication, and fingerprint-based user authentication.

12. The electronic device of claim 10, wherein the first interface for iris-based user authentication further comprises a graphic of the user's eyes.

13. The electronic device of claim 11, wherein the processor conducts other user authentication during the iris-based user authentication.

14. A method for operating an electronic device which comprises an iris scanning sensor, comprising:
detecting a display-on event in a display-off state; and
when the display-on event is a user intended input, activating the iris scanning sensor.

15. The method of claim 14, wherein activating the iris scanning sensor comprises:
when the display-on event is an intended user input, displaying a first user interface which guides iris recognition.

16. The method of claim 15, wherein the iris scanning sensor comprises:
an Infrared (IR) camera and a light source, and
activating the iris scanning sensor further comprises:
while displaying the first user interface, determining to activate the IR camera or the light source.

17. The method of claim 14, further comprising:
when the display-on event is an unintended user input, displaying a second user interface prompting the user for an input requesting iris recognition.

18. The method of claim 17, wherein the iris scanning sensor comprises:
an IR camera or a light source module, and further comprising:
while displaying the second user interface, activating the iris scanning sensor after receiving the input requesting iris recognition.

19. The method of claim 14, wherein the intended user input comprises at least one of button selection, touch input detection, and cover case opening in the electronic device.

20. An electronic device comprising:
a touchscreen;
an infrared camera; and
a processor operably coupled to the infrared camera and the touchscreen, the processor configured to:
when the touchscreen is de-illuminated, detect one of a button selection, a touch on the touchscreen, and receipt of a push notification;
causing the touchscreen to illuminate responsive to detecting one of a button selection, a touch on the touchscreen, and receipt of the push notification and display a locked screen;
when detecting the button selection or the touch on the touchscreen, automatically activating the infrared camera; and
when detecting the push notification, displaying a prompt on the touchscreen for a user input requesting iris detection.
